(19) United States
(12) Patent Application Publication
(10) Pub. No.: US 2004/0047804 A1
(43) Pub. Date: Mar. 11, 2004

(54) Enhanced radiation therapy

(75) Inventors: Gerald L. Wolf, Westboro, MA (US); Gregory L. McIntire, West Chester, PA (US); Edward R. Bacon, Audubon, PA (US)

Correspondence Address: FISH & RICHARDSON PC, 225 FRANKLIN ST, BOSTON, MA, 02110, US

(73) Assignee: The General Hospital Corporation, a Massachusetts corporation

(21) Appl. No.: 10/639,915

(22) Filed: Aug. 12, 2003

Related U.S. Application Data
(63) Continuation of application No. 09/585,938, filed on Jun. 2, 2000, now abandoned.
Application No. 09/585,938 is a continuation of application No. 09/183,166, filed on Oct. 29, 1998, now abandoned.

(51) Int. Cl. A61K 51/00

(52) U.S. Cl. 424/1.11

(57) ABSTRACT

The invention features new methods of enhanced radiation therapy based on the discovery that by using controlled combinations of (i) specific radiodense compositions, (ii) specific modes of administration of these radiodense compositions, and (iii) specific energy bands and sources of radiation, that the effect of radiation on tumors and other diseased tissues can be effectively and safely enhanced to provide significantly improved radiation therapy.

### CROSS REFERENCE TO RELATED APPLICATIONS

This application is a continuation of, and claims priority from, U.S. patent application Ser. No. 09/585,938, filed on Jun. 2, 2000, which is a continuation of U.S. patent application Ser. No. 09/183,166, filed on Oct. 29, 1998. The contents of both applications are incorporated herein by reference in their entirety.

### FIELD OF THE INVENTION

The invention relates to new methods of enhancing radiation therapy, e.g., for tumor therapy.

### BACKGROUND OF THE INVENTION

Radiation therapy has been used with some success in treating tumors and other diseases. However, the dose of effective radiation must be sufficiently limited to the tumor or other target tissue to avoid injuring the surrounding tissues and the overall health of the patient.

Some efforts have been made to enhance the absorption of radiation by tumors compared to normal tissues adjacent to the tumor and throughout the body. For example, it has been shown that the presence of iodinated x-ray contrast agents within animal tumors during treatment with an external computed tomographic (CT) device operating in the orthovoltage range can somewhat improve treatment response.

### SUMMARY OF THE INVENTION

The invention is based on the discovery that by using controlled combinations of (i) specific radiodense compositions, (ii) specific modes of administration of these radiodense compositions, and (iii) specific energy bands and sources of radiation, that the effect of radiation on tumors and other diseased tissues can be effectively and safely enhanced to provide significantly improved radiation therapy.

In general, the invention features a method of treating a target tissue, e.g., a tumor, in a patient by administering to the patient systemically a radiodense composition including a small molecule radiodense material in an amount sufficient to accumulate selectively within the target tissue compared to non-target tissue; and inserting a radiation emitting source, e.g., a probe or radiopharmaceutical, into the target tissue and irradiating the target tissue from within for a time and under conditions sufficient to kill cells within the target tissue. For example, the radiodense composition can accumulate selectively at the outer edge of the target tissue, and/or penetrate into the tissue.

In specific embodiments, the radiodense composition is administered intravenously as a bolus, followed by an infusion of the same or a different radiodense composition at a rate that equals the blood clearance rate of the first radiodense composition. The radiodense composition can be iohexol, iopamidol, ioversol, ioxilan, iomeprol, or iodixanol.

In other embodiments, the amount of the radiodense composition administered is sufficient to increase the radiation absorption of the outer edge of the target tissue by at least 10, 50, 100, or 200 Hounsfield units (HU), or more, e.g., 300, 500, or 1000 HU, the radiation can have an energy of less than 140 kiloelectron volts, e.g., about 20 to 80, or 40, kiloelectron volts, or more than 1.02 megaelectron volts, e.g., 5, 10, or more megaelectron volts, and the radiodense composition can be linked to a targeting agent that binds specifically to the target tissue.

In another aspect, the invention features a method of treating a target tissue, e.g., a tumor or diseased skin or a lymph node, in a patient by administering to, e.g., into, the target tissue, e.g., by direct injection or by painting the composition onto the skin, an amount of a radiodense composition; and irradiating the target tissue with an external radiation source emitting radiation at an energy of less than 140 kiloelectron volts (Kev), e.g., at 20, 40, or 80 Kev, or more than 1.02 megaelectron volts (Mev) for a time and under conditions sufficient to kill cells within the target tissue. For example the radiodense material can be administered to the target tissue in a stent implanted within or adjacent to the target tissue.

In specific embodiments, the amount of the radiodense composition is sufficient to increase absorption of radiation in the target tissue by at least 10 or 200 HU, or more, e.g., 500, 750, or 1000 HU or more. These units can be measured by methods described herein.

In certain embodiments, the radiodense composition includes a mixture of a small molecule radiodense material and a large molecule radiodense material (or just includes a large or small molecule material), and the radiodense composition can include iodine, barium, bismuth, boron, bromine, calcium, gold, silver, iron, manganese, nickel, gadolinium, dysprosium, tungsten, tantalum, stainless steel, or nitinol, or a combination of any one or more of the above. The radiodense composition can also be a radiodense material present within a small, lipid soluble molecule, such as ethiodol (Lipiodol™), which is poppy seed oil in which carbon atoms are iodinated.

In other embodiments, the radiodense composition has a dwell time within the target tissue of at least 3, 5, 10, 15, 20, or 24 or more hours. Certain compositions can be designed to have dwell times of several days to weeks. In specific embodiments, the radiodense composition can be about 10 nanometers to 100 microns in size, and can be NI-243, NI-212, or a liposome comprising iohexol (CTP-10, Nycomed, Wayne, Pa.).

In another aspect, the invention features a method of treating a diffuse tumor, e.g., a metastatic tumor, in a patient by administering to the patient systemically a radiodense composition that includes a small molecule radiodense material in an amount sufficient to accumulate selectively within the diffuse tumor tissue compared to non-tumor tissue; and irradiating the body part of the patient in which the diffuse tumor is located with radiation for a time and under conditions sufficient to kill cells within the diffuse tumor. For example, the radiodense composition can accumulate selectively at the outer edge of the tumor and enter and accumulate within the tumor tissue, and can be administered intravenously as a bolus, followed by an infusion of the same or a different radiodense composition at a rate that equals the blood clearance rate of the radiodense composition.

In other embodiments, the radiation can have the energy levels described above, and the radiodense composition can be linked to a targeting agent that binds specifically to the target tissue, and can be a particle having ranging in size from, e.g., 30 to 300 nanometers.

Radiodense compositions can be or include small molecules of radiodense materials, which are less than 1 nanometer in size and diffuse readily in aqueous spaces of the body. Although these small molecules may not penetrate biological structures such as most cell membranes or tight endothelial junctions as found the capillaries of brain, retina, or testis, they do penetrate capillary walls in most other parts of the body, e.g., the capillaries feeding tumors.

Radiodense compositions can also be or include large molecules of radiodense materials, which have a much lower diffusion rate than the small molecules, and which do not generally penetrate normal blood capillaries in either transport direction, i.e., from blood to tissue or from tissue to blood. So considered, these large molecules are generally larger that about 10 to 20 nanometers, and can be 100 to 400 nanometers in size, and can be up to several hundred of microns in size, e.g., 100, 300, 500 or more microns. Large molecules can include liposomes, esters, polymers, and emulsions as described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The new methods provide numerous advantages. For example, the new methods enable shorter therapeutic regimens and expand treatment options. The new methods also enable the application of prophylactic radiation of vascular stents with orthovoltage or megavoltage equipment, and enable the use of low energy x-rays (e.g., 20 Kev to 140 Kev) for tumor therapy by increasing the efficacy of the treatment itself. Thus, the new methods spare normal tissue from unnecessary radiation. Furthermore, it is expected that more than half of all radiation treatments of cancer can be improved by the new methods.

In addition, after injection of the new radiodense compositions, the local and any systemic distribution of the composition can be visualized in the patient using standard x-ray techniques, and therapy is carried out only if the resulting distribution is favorable.

Other features and advantages of the invention will be apparent from the following detailed description, and from the claims.

### BRIEF DESCRIPTION OF THE DRAWINGS

FIG. 1 is a schematic diagram of radiation treatment planning.

FIG. 2 is a schematic diagram of tissue density from CT scanning.

FIG. 3 is a graph comparing the change in radiation absorption in different parts of an adenocarcinoma injected with a radiodense composition (Omni-350).

FIG. 4 is a graph comparing the change in radiation absorption in different parts of a glioma injected with a radiodense composition (NI-243).

FIG. 5 is a graph comparing the change in radiation absorption in different parts of an adenocarcinoma injected with a radiodense composition (NI-212).

FIG. 6 is a graph showing the effect of radiation dosage on the survival of tumor cells (V79) in vitro in the presence of different radiodense compositions.

### DETAILED DESCRIPTION

The invention relates to the combination of specific radiodense compositions, specific modes of administration of the radiodense compositions, and specific energies and sources of radiation, to provide a significant increase in the selective absorption of radiation in tumors and other diseased tissues to provide greatly enhanced methods of radiation therapy.

The radiodense compositions act as adjuvants and enhance, i.e., improve the toxic effect of, radiation therapy at locations where the composition and the radiation coexist in the proper dosage range. There is a nonintuitive relationship between the formulation and administration regimen of the radiodense composition and the external or internal radiation source as described in further detail below.

The physiology of tumors restricts the entry of certain materials from the blood. The same barrier, however, restricts the exit of molecules injected into the tumor. If the injectates contain sufficient high Z materials, they will change both the attenuation and absorption of radiation in proportion to their local change in electron density. However, the injected materials have to be present at the time the radiation is delivered. For this reason, longer lasting injectates are desirable to avoid new injections yet enable repeated, or long duration administrations of radiation.

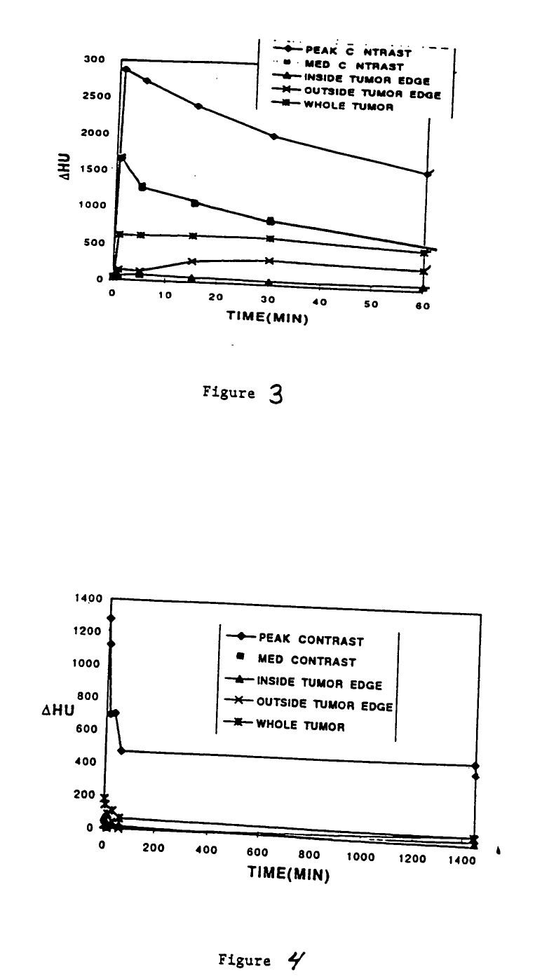

The new methods provide for the selective increase in the level or concentration of radiodense compositions within tumors by factors of 10 to 20 or from 50 to 3000 HU (as shown in FIGS. 3 to 6) as compared to the surrounding normal tissues, and thus provide a commensurate increase in the absorption of radiation within treated tumors. The radiodense compositions remain within the target tumors following intratumoral administration for periods of hours, days, or weeks, depending upon their formulation.

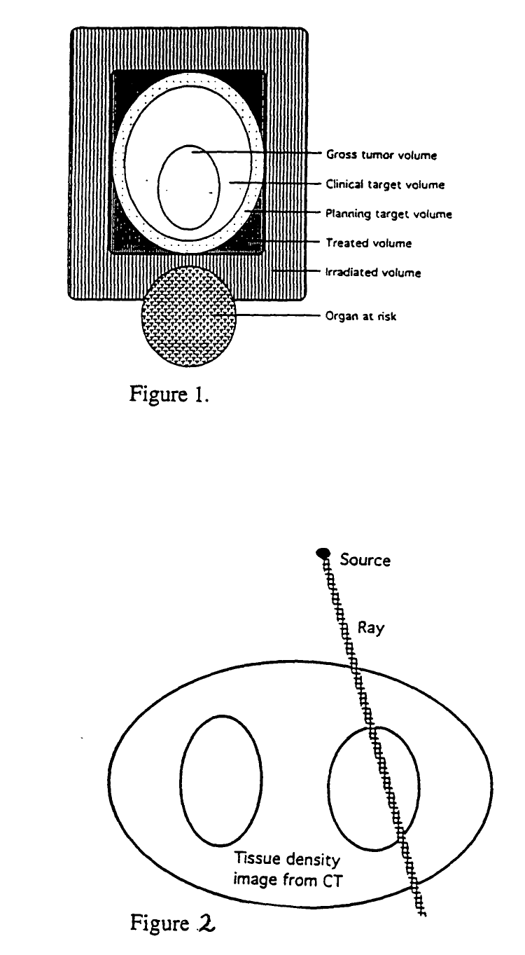

It is common to use computed tomography (CT) to identify the structures of interest. CT, at its physical core, plots the geometric distribution of electron density. It is the interaction of radiation with local electrons that provides the toxic (cell killing) effect of orthovoltage radiation. Thus, the CT absorption differences of the tumor versus the normal tissues defines where the radiation energy will be absorbed. FIG. 1 shows the aspects of conventional radiation therapy targeting, while FIG. 2 shows a schematic CT where two ovoid regions have increased radiodensity (higher HU). The difference between the more radiodense tissues and the less radiodense tissues, again in HU, directly reflects differences in electron density and the proportionate differences in radiation absorption at this incident energy in the orthovoltage range.

As discussed in further details in the Examples below, FIGS. 3-5 show graphs where the relative radiodensity is plotted versus time for various regions within experimental tumors. These examples are selected to show a mixture of small and large molecule radiodense materials (NI-243 and NI-212), and a small molecule only (Omni-350).

The clinical drawback of unenhanced CT is that nearly all soft tissues, normal and tumorous alike, have similar electron density, and thus the same x-ray absorption. Conventional 3D radiation treatment planning is complicated by this lack of any useable differences in the absorption of radiation between tumors and the surrounding healthy tissues.

The radiodense compositions described herein can be tailored to various types of radiation therapy, and can be used with standard external radiation sources, or with new internal radiation sources (such as the Photoelectron Corp. radiosurgery probe system) as well as brachytherapy radiopharmaceuticals that emit radiation from within the tumor.

While the new methods for enhancing radiation therapy will be applicable to any solid tumor, it is envisioned that they are especially useful for treating prostate, breast, lung, head and neck, brain, and liver tumors. In addition, the new methods should be useful for enhancing alternative radiation procedures where the disease is not cancer, but involves a tissue that can selectively absorb or bind to the radiodense compositions.

Radiodense Compositions

The radiodense compositions are designed to selectively increase the concentration or level of one or more radiodense (electron dense) agents or materials, such as iodine, in a target tissue, e.g., a tumor, immediately upon administration, and to maintain the elevated level for the time required to deliver one or more radiation treatments, e.g., several hours, days, or weeks after the initial administration.

A radiodense composition is a material that contains more electrons and/or more atomic nuclei per volume than are contained in the soft tissues of animals or man. Generally, the material includes elements with a high Z number, which increases both electron and nuclear density. Such high Z materials include iodine, barium, bismuth, boron, bromine, calcium, gold, silver, iron, manganese, nickel, gadolinium, dysprosium, tungsten, tantalum, stainless steel, nitinol, or any other material that absorbs incident radiation.

By increasing the level of radiodense compositions in a tumor, the compositions provide a significant enhancement of radiation absorption, thereby increasing the deposition of radiation energy in the tumor and maximizing the therapeutic effect at the target. As an added benefit, that radiation that is absorbed within the tumor is not available to cause damage in the normal tissues lying beyond the tumor.

The radiation absorbing radiodense composition can be the element itself, or can include the high Z element incorporated into other chemical carriers to obtain useful or improved pharmacokinetics, safety, or cost. Suitable materials include, for example, small molecule, water-soluble or lipid-soluble, iodinated agents (Table 1); insoluble iodinated derivatives with sizes from 0.050 to 50 microns; encapsulated agents such as liposomes; and micelles composed of block co-polymers. Other materials that absorb radiation and selectively accumulate in a target tissue are also useful in the new methods. In some applications, mixtures of radiodense materials may have a particular utility.

Radiodensity can be calculated from the nature of the radiodense material. The pharmacokinetics of electron dense materials are often conveniently determined with spatially and temporally resolved computed tomography (CT) where the relative electron density is expressed in Hounsfield Units (HU). In this case the local radiodensity can be readily compared to water or nonenriched soft tissues. A Hounsfield scale where air is −2000 HU, water is zero HU, and bone is +4000 HU is used herein.

The level of radiodense enrichment (the radiation absorption enhancement) of a tissue can be calculated in HU by:

1. First scanning the region of the body containing the target tissue with a CT scanner operating at a set orthovoltage such as 140 KeV;

2. Administering the radiodense composition in the desired dose, rate, and location;

3. At any time thereafter, repeating the CT scan;

4. Determining the x-ray absorption in HU of each region of interest;

5. Subtracting the value obtain with the first scan from any timed value obtained later; and

6. Determining the subtracted value in HU or (ΔHU in FIGS. 3-5), which is directly related to the increased electron density in the region of interest at the time of the post-administration scan.

Since the composition of the radiodense composition is known, the increment in electron density of any tissue at any time is directly proportional to the increment in nuclear density in the same region at the same time. In fact, the ΔHU value can be directly converted into concentration of the added radiodense composition in mg per volume of tissue by constructing a standard curve. This can be done by placing samples containing different known amounts of the radiodense composition in the scanner and determining the HU for each concentration; and plotting the ratio between concentration and HU. The slope of this ratio is the correction factor that can be used to correct the ΔHU values for each tissue directly to concentration of the administered radiodense composition.

The radiodense compositions can include individual radiodense materials, e.g., a “small molecule” or “large molecule” radiodense material (as described herein), or the compositions can include mixtures of two or more radiodense materials, e.g., a mixture of different small or large molecules, or a mixture of small and large molecules. The combination of small and large molecules provides a radiodense composition that rapidly increases the level of the small molecule (and large molecule if injected into the target) radiodense material in a given target tissue, e.g., a solid tumor, to a high density, and then provides a sustained level of the large molecule radiodense material for several days to weeks.

The small molecule radiodense materials are on the order of one nanometer or smaller in size, and are typically water soluble. Thus, these materials quickly diffuse into and throughout a tumor or the bloodstream to rapidly increase the level of the radiodense material within the target tumor. The rapid diffusion is also a limitation, because these materials diffuse out of the tumor into the bloodstream within about three to four hours or so, depending on the specific size and composition. Thus, small molecule radiodense materials can be used alone in radiodense compositions only under certain circumstances as describe herein.

The small molecule radiodense materials are selectively taken up by tumors compared to healthy tissues because tumors have greater blood perfusion than healthy tissue, and because tumors generate cytokines such as vascular endothelial growth factor that make the blood vessels that feed the tumor “leaky” to allow for a faster and greater exchange of materials between the blood vessels and the tumor compared to normal tissues.

Exemplary small molecule radiodense materials suitable for use in the new methods are listed in Table 1, and include iohexol (Omnipaque™), Hypaque™, and iodixol (Visipaque™). The materials listed in Table 1 are commercially available.

TABLE 1
Small Molecule Radiodense Materials

Ionic Agents (all concentrations)
Diatrizoates
Hypaque ™, Angiovist ™, MD-60 ™, MD-76 ™, Renografin ™,
Renoca ™, Reno ™, Renovist ™, Urovist ™
Iothalamates
Conray ™, Angio-Conray ™, Cysto-Conray ™, Cysto-ConrayII ™,
Vascoray ™
Iodamides
Renovuer ™
Ionic-Nonionic Agents
Ioxaglate
Hexabrix ™
Nonionic Monomers
Iohexol
Omnipaque ™
Iopamidol
Isovue ™
Ioversol
Optiray ™
Metrizamide
Amipaque ™
Nonionic Dimers
Iodixanol
Visipaque ™
Lipid-Soluble Agents
Ethiodol
Lipiodol ™
Biliary Agents
Iodoxamate
Cholovue ™
Iodipamide
Chlorografin ™, Telepaque ™

The large molecule radiodense materials are designed to have a much longer dwell time in the target tissue. Accordingly, these large molecules are less water soluble and diffuse much more slowly than the small molecules, and are too large to pass easily into or out of the bloodstream or from the tumor into the blood. As a result, they provide a dwell time of one or more days to weeks once injected into a tumor. These large molecule radiodense materials are on the order of 100 to 400 nanometers, or even larger in size, and can be up to several tens or hundreds of microns, in size.

Examples of the large molecule radiodense materials include water-insoluble esters of diatrizoic acids. The esters of these diatrizoic acids are combined into large, solid conglomerates that are then milled or ground into small uniformly sized particles of, e.g., 100 to 300 nm.

Particularly suitable large molecules include WIN 8883 [ethyl-bis(3,5-acetylamino)-2,4,6-triiodobenzoate; Sterling] which is a water-insoluble nanoparticulate of diatrizoic acid ester about 300 nm in size. Other useful large molecule radiodense materials include NC 70146 [1-(ethoxycarbonyl)pentyl-bis (3,5-acetylamino)-2,4,6-triiodobenzoate; Nycomed, Wayne, Pa.], NC 67722 [6-(ethoxycarbonyl)hexyl-bis (3,5-acetylamino)-2,4,6-triiodobenzoate; Nycomed]; and NC 12901 [(ethoxycarbonyl)methyl-bis (3,5-acetylamino)-2,4,6-triiodobenzoate]. All of these compositions are insoluble in water and are milled to the desired particle size. They differ in their ease of hydrolysis in the body and in their metabolism. Other large molecule radiodense materials include gadolinium oxide, gadolinium oxalate, manganese doped hydroxyapatite.

Additional large molecule radiodense materials include liposomes that encapsulate or entrap radiopaque agents, or that include radiopaque agents in the external phase (i.e., continuous solution phase). For example, the radiopaque agent CTP-10 (iohexol) can be encapsulated within a liposome using standard techniques. The size of the liposomes can be about 10 to 400 nanometers. The radiodense material can be water-soluble and present at a high concentration within the liposome and in equal concentration in the membrane of the liposomes.

Examples of radiodense compositions that include both small and large molecule radiodense materials are NI-212 (Nycomed) and NI-243 (Nycomed). NI-212 is an insoluble triiodinated ester (listed above) and contains water soluble iohexol (Omnipaque™). NI-243 is another insoluble triiodinated ester (listed above) with its first soluble metabolite sodium 6-[3,5-bis(acetylamino)-2,4,6 triiodophenyl)carbonyloxy]hexanoate (small molecule, NC 68056) which is the corresponding carboxylic acid derived from the loss of the ethyl ester.

Modes of Administering Radiodense Compositions

The radiodense compositions can be administered directly into a solid tumor, administered systemically to contact the surface and permeate into the interior of a solid tumor from the outer surface, or administered systemically to permeate rapidly into small, diffuse, e.g., metastatic, tumors. The mode of administration depends upon the type of tumor, the nature of the radiodense composition, and the type and source of the radiation to be employed for therapy.

When treating a solid tumor using an external source of orthovoltage or megavoltage radiation, such as a CT scanner, it is advisable to administer the radiodense compositions intratumorally, e.g., by direct injection (e.g., using a small gauge needle of the appropriate length). To the extent that the treatment will require long or repeated exposures, a large molecule radiodense material with a long dwell time should be included. However, the addition of a small molecule radiodense material can give a “boost” to that region of the tumor accessed by the diffusion of the small molecule and very significant radiation enhancement can be achieved shortly after intratumoral administration of a mixed small and large molecule composition. As tumor size increases, it may be desirable to deposit the radiodense composition at several central locations in the tumor.

This allows the radiodense composition to diffuse and migrate within the tumor from the inside towards the outside, with the large molecule material remaining at or near the site of injection, and the small molecule material moving outwards from the site of injection, creating a gradually decreasing electron density (concentration gradient of the radiodense material) within in the tumor from the highest in the center to the lowest at the edges of the tumor. Thus, the combination radiodense compositions are ideal for use with an external radiation source, which provides an energy profile within the tumor that is the most intense at the outer edge of the tumor, and which gradually decreases in intensity towards the center of the tumor.

As a result, the radiodense composition enhances the absorption (and thus killing power) of the radiation most in the center of the tumor, where the radiation intensity is lowest, and gradually decreases the enhancement towards the outer surface of the tumor, where the least enhancement is required (because the radiation intensity is the highest). This provides the optimal radiation dosage (absorption) throughout the tumor, and can be tailored to specific sizes and types of tumors by adjusting the ratio of small to large molecule materials, the radiation dosage, and the timing and length of radiation administration.

Of course, a sufficient amount of the radiodense composition is administered into the tumor to ensure a certain minimal level of enhancement at the surface or outer edge of the tumor to allow the tumor to selectively absorb a greater amount of radiation than neighboring healthy tissue.

On the other hand, when treating a solid tumor with an internal radiation source, the radiation is most intense in the immediate vicinity of the source, e.g., in the center of the tumor if the source is inserted into the center. Therefore, the radiation requires the most enhancement at the surface or edge of the tumor, and the concentration of the radiodense composition should gradually decrease to the lowest level at the center of the tumor. Suitable internal radiation sources include small radioprobes, such as a radiosurgery probe manufactured by Photoelectron Corp. (Lexington, Mass.), and brachytherapy implants of solid or liquid radioactive materials. Such implants can include solid or encapsulated radiopharmaceuticals such as P-32, Sc-47, Co-60, Cu-67, Sr-89, Y-90, Rh-105, 1-131, 1-125, Sm-153, Lu-177, Re-188, Ir-194, Au-199, Ra-226, Rn-222, and Am-241.

In this setting, e.g., when using a radiosurgery probe operating in the low kiloelectron voltage range, the radiodense composition should include mostly small molecules that are administered systemically to diffuse into the tumor, which ensures a gradually decreasing concentration gradient of the composition from the outer surface or edge to the center of the tumor, and thus a gradually decreasing level of enhancement of the radiation absorption from the outer surface to the center, which corresponds inversely to the radiation intensity profile emitted by the source centered in the tumor. Again, this provides the optimal radiation dosage throughout the tumor, and can be tailored to specific sizes and types of tumors and radiation sources. Due to their safety, nonionic small molecule radiodense materials such as iohexol, iopamidol, ioversol, ioxilan, and iodixanol are suitable.

When the small molecules are administered systemically adjustments should be made for the rate at which the radiodense materials diffuse out of or are cleared from the bloodstream (e.g., into the target such as a solid tumor). For example, the radiodense compositions can be given as an intravenous bolus with a subsequent infusion equal to the blood clearance rate of the composition to sustain the desired concentration. The bolus dose should be sufficient to increase the edge region of leakage by 10 to 200 Hounsfield units, and the bolus and/or infusion must sustain the edge enhancement for the duration of treatment, e.g., 0.5 to 3 hours.

For other internal radiation sources, the radiodense compositions can be administered as above, taking into consideration the radiation dosimetry for the particular source. For example, the energy level emitted by the specific radiopharmaceutical should be determined, and the radiodense composition chosen accordingly. For example, 1-125 and Am-241 emit in the orthovoltage range, while Ra-226, Rn-222, and Y-90 emit in the megavoltage range. The radiodense materials described herein all increase both electron density and nuclear density because they contain high Z materials. Thus, they can be used with radiation sources that emit in the orthovoltage and megavoltage ranges, as well as the midrange, as described in further detail below.

In yet another scenario, if the tumor to be treated is a diffuse and/or metastatic tumor, then the radiodense composition should include only small molecule radiodense materials, and should be administered systemically to provide radiodense enhancement due to the leakiness of such tumors and their large extracellular space that can be loaded with the radiation enhancer. The radiation is then administered to the part of the body know to harbor the metastatic tumor or to the body region at risk of metastatis in the case of “prophylactic” radiation therapy.

In general, when the radiodense compositions are administered systemically, tumors that are more “leaky” will require either a lower concentration of the radiodense composition, and/or a lower radiation dose, while less leaky tumors will require a higher systemic concentration of the radiodense composition, or a longer duration in the bloodstream (e.g., maintained by infusion) to allow sufficient accumulation within the tumor. The specific therapeutic regimen of radiation and systemic administration of a particular radiodense composition can be determined using modern imaging and temporal and spatially quantitative methods (such as functional computed tomography) and radiation simulation.

Certain radiodense compositions are designed to be targeted to specific parts of the body, e.g., by naturally accumulating selectively in the kidneys, lymph nodes, and/or liver. These compositions are of a size and material, and are administered in a way, that induces the selective accumulation.

For example, radiation treatment of disease, usually neoplasia, in the lymph nodes can be significantly enhanced using radiodense materials that are naturally accumulated selectively in the lymph nodes (to both neoplastic and healthy tissue) by the body. Large molecule radiodense materials with a particle size of about 30 or 50 to 300 nanometers (with or without a small radiodense water soluble molecule) can be administered percutaneously to the interstitial site that supplies the lymph to the target lymph node, e.g., by using methods describe in, e.g., U.S. Pat. Nos. 5,111,706 and 5,496,536. The target lymph node accumulates the radiodense material selectively with radiation absorption enhanced by 100 HU to often more than 400 HU. When then exposed to either external or internal radiation, the radiodense material in the target lymph node will enhance radiation damage to the node and its contents while allowing lower radiation doses to the surrounding, uninvolved tissues.

This method has special utility for the sentinel nodes of great clinical interest in breast cancer and melanoma, where the location of these nodes can be determined by diagnostic lymphography, and therapy then directed at the small body region containing the target node or nodes. Other deep nodes such as those in the thorax, neck and abdomen could be similarly enhance with radiodense adjuvants and treated with radiation therapy with less damage to nearby structures. Diffuse processes involving lymph nodes such as lymphoma and Hodgkins disease, can also be targeted with the same methods and materials. Large radiodense molecules of the type describe above also naturally and automatically target organs rich in macrophages, such as the liver and spleen, following systemic administration. Thus, the new methods are especially effective when used to treat tumors that are located in one of these naturally targeted body locations or organs, if the radiodense compositions are designed to take advantage of this natural targeting mechanism.

The radiodense compositions can also include known targeting agents that will home within the body to selected targets, such as tumors or other diseased tissue. For example antibodies, e.g., monoclonal antibodies, that bind specifically to tumor surface antigens can be linked (e.g., by covalent, non-covalent, ionic, or by nonionic bonds) to the radiodense materials. Alternatively, numerous binding pairs, e.g., streptavidin/biotin, are known that can be used in the present methods. For example, biotin can be linked to a monoclonal antibody that binds specifically to the surface of a tumor. Streptavidin is linked to a radiodense composition, and the complex is administered systemically. The very high binding specificity between biotin and streptavidin provides a very selective and powerful targeting mechanism for the radiodense composition.

In addition, there a various polymers and long-chain compounds that are known to accumulate selectively in the kidneys, lymph nodes, and/or liver. These compounds can also be linked to the radiodense compositions to provide a targeting to tumors in these organs. For example, compounds that bind specifically to mannose receptors on liver cells, can be used to target the radiodense compositions when treating liver cancer.

The new methods can be used to treat tissues other than neoplastic tissues. For example, the methods can be used to treat excessive local cell proliferation in other clinical circumstances where such proliferation is harmful. Radiation damage is known to be cell cycle dependent and non-neoplastic cells that are proliferating have increased susceptibility to damage by radiation. However, damage to non-target cells must, and can be avoided using the new methods.

Such a circumstance occurs following vascular intervention such as balloon angioplasty or bypass surgery using natural or artificial grafts. As a response to the local trauma, cells proliferate beginning a few days following intervention and may proliferate to the point where the volume of proliferated cells can jeopardize the vascular lumen. A partial solution to this problem has been to place a metallic stent across the traumatized vascular surface, but cell proliferation can still occur with migration through the stent. However, according to the new methods, if the stent includes a radiodense material as described herein, and is located immediately adjacent to the zone where proliferation will subsequently occur, the radiodense material will enhance absorption of radiation in the target region. Thus, when the stent is irradiated (with an external or internal source), restenosis will be reduced and adjacent non-target tissues will be spared.

The new methods can also be used to treat external tissues, such as the skin in diseases like acne or psoriasis, or skin tumors like melanomas. In these cases, the radiodense compositions are “painted” on the target tissue (e.g., mixed with an agent that enhances skin permeability, such as DMSO) prior to radiation therapy.

All of the radiodense compositions described herein can be administered either alone or together with conventional oncology therapeutic drugs. Moreover, it should be clear from the foregoing that the new methods require knowledge of the pharmacokinetics of the radiodense material in the composition in addition to the radiation dosimetry usually considered for the radiation regimen.

The new methods also provide an important safety benefit, in that radiation will not be administered to a patient if the radiodense compositions do not diffuse throughout the tumor, or leak out of the tumor (when injected directly), or accumulate in the tumor (when administered systemically) as planned. This is possible, because the distribution of the radiodense compositions can be determined with quantitative imaging prior to therapy with, for example, CT. Thus, the amount of radiation enhancement, its duration, and the expected response can be accurately modeled. For example, if a radiodense composition is administered systemically, the tumor can be imaged at short intervals to determine exactly when a sufficient concentration of the composition has accumulated within the tumor. This regimen can be repeated when the radiation is to be administered. In the case of diffuse and/or metastatic tumors, only one specific portion of the tumor needs to be imaged, as all the other portions of the tumor will accumulate the radiodense composition in a similar manner.

Radiation Dosages and Methods of Irradiation

Radiation is useful for killing tumor cells by inducing irreparable damage to the genome; normal cells are equally damaged but may have a slight repair advantage. Rarely is such radiation absorbed as a single and total exchange of energy between the beam and a locus on a gene. Instead, radiation absorption is usually through multiple interactions with tissue, often scattered over some distance. The radiation source can be either external or internal. For radiation therapy, the useful energy ranges are from 20 Kev to tens of Mev.

The dominant mechanism of absorption of radiation is fairly well understood and varies with the incident energy ranges as shown in Table 2. There are three general energy ranges listed in this table known as “orthovoltage,” “midrange,” and “megavoltage.” Although the three ranges overlap somewhat, it is convenient to use these ranges for discussion with respect to the new methods.

The lowest energy (orthovoltage range) is efficiently absorbed by resident electrons in proportion to their abundance. Some K-shell electrons are more efficient at selected energies, but this effect is not prominent with normal tissue composition. As Table 2 shows, absorption of this energy range decreases with the inverse of incident energy cubed, but increases with atomic number cubed (and electron density). Orthovoltage has poor penetration depths and lots of scatter.

The midrange of radiation energy is mostly limited to radioactive materials and brachytherapy applications.

The megavoltage range is currently the most useful due to enhanced depth of penetration and little increased absorption with moderate Z number radiodense materials such as present in bone (calcium phosphate salts). However, above 1.02 Mev, the dominant mechanism of absorption is pair production due to interaction with the atomic nucleus. As Table 2 shows, this interaction increases with energy, Z number, and nuclear density.

TABLE 2

| | | Dominant | Broad |
|---|---|---|---|
| Energy | Description | Mechanism | Patterns |
| 20 to ≈250 | Orthovoltage | photoelectron | 1/E³ |
| Kev | | “bound electrons” | Z³ times Z |
| | | | # electrons times |
| | | | density |
| ≈200 Kev | Midrange | Compton and | 1/E |
| to 1 Mev | | coherent Free | density matters, Z |
| | | electrons | does not |
| ≧1.02 Mev | Megavoltage | Pair production | Z × Z × E |
| | | atomic nucleus | nuclear cross-section |
| | | | & density increases |
| | | | with energy |

Table 3 below shows hypothetical calculations of the relative absorption of radiation (targeting ratio) where the tumor target has been increased by 1000 HU with a radiodense material that increases the tissue density from 1.004 to 1.203 such as might occur with a large molecule radiodense material. This simulation shows the large increments of radiation absorption enhancement in the target in the orthovoltage and megavoltage range. Due to the enhancement of tissue density, there is a measurable benefit even in the midrange.

TABLE 3

| Energy (Mev) | Unenhanced | Enhanced | Targeting ratio |
|---|---|---|---|
| 0.1 | 0.1566 | 1.753 | 12.2 |
| 0.15 | 0.1375 | 0.646 | 5.7 |
| 0.20 | 0.1245 | 0.339 | 3.73 |
| 0.5 | 0.0874 | 0.0966 | 2.10 |
| 1.0 | 0.0639 | 0.0589 | 1.92 |
| 5.0 | 0.0279 | 0.0360 | 2.29 |
| 10.0 | 0.0210 | 0.0394 | 2.88 |
| 20.0 | 0.0178 | 0.0467 | 3.62 |
| 50.0 | 0.0179 | 0.0597 | 4.51 |
| 100.0 | 0.0179 | 0.06893 | 4.87 |

### EXAMPLES

The invention is further described in the following examples, which do not limit the scope of the invention described in the claims.

### Example 1

### Formulation of a Large Molecule Radiodense Composition

Formulations suitable for use in the new methods can be prepared from an insoluble iodinated material that is subsequently milled to provide the desired size as described, e.g., in U.S. Pat. Nos. 5,322,679 and 5,318,767. For example, NC 67722 (6-ethoxycarbonyl)hexyl-bis(3,5-acetylamino-2,4,6 triiodobenzoate; Nycomed, Wayne, Pa.) can be the starting insoluble iodinated material. The 67722 suspension can be wet milled using water soluble iodinated agents such as iohexol as an auxiliary wetting agent. Milling is continued until the desired particle size of 67722 is obtained, e.g., about 100 to 300 nm. The resulting mixture can then be diluted with a presterilized stock solution of Pluoronic F98 (BASF, Parsippany, N.J.) and glycerol and the final pH adjusted.

If the water soluble wetting agent is NC 69056, a final composition of 15% (wt/vol) NC 67722, 3% NC 68056, 3% F98, and 1.75% glycerol is obtained with an iodine concentration of 180 mg I/ml. This formulation, designated NI-244, is suitable for lymph node and intratumoral applications where the average particle size is 97 nanometers as determine with a light scattering device (Horiba, model 910).

Other starting materials can be used to prepare other radiodense compositions using similar methods. These compositions can be coated with surfactants, and can range in size from about 0.05 to 50 microns. Insoluble materials that have been used to prepare radiodense compositions include NC 12901, NC 70146, and WIN 8883 as identified above. The insoluble iodinated agents can also be prepare without the water soluble iodinated agent to provide a composition containing only large radiodense molecules.

### Example 2

### Large Molecule Radiodense Compositions with Added Therapeutic Ingredients

Numerous patents describe nanoparticulates of drugs. See, e.g., U.S. Pat. No. 5,145,684. As solids, these materials are somewhat more dense than soft tissues and may be targets for radiation therapy, thereby combining local drug efficacy with radiodense properties useful for radiation therapy. However, since the starting materials are often insoluble particulates, they can be mixed with the insoluble iodinated materials to generate a composition that is imageable, has local drug efficacy, and serves as a target for radiation therapy in the new methods.

### Example 3

### Combinations of Small and Large Molecule Radiodense Materials

Various radiodense compositions have been prepared that include both small molecule radiodense materials and large molecule radiodense materials. For example, NI-243 combines Win 8883 with water soluble iohexol; NI 212 combines NC 70146 with iohexol; and NI-244 combines NC 67722 with NC 68506 in ratios of approximately 5 parts large molecule to 1 part small molecule (wt/vol). These ratios are selected depending upon their intended purpose. In particular, it is easy to add more water soluble small molecule such as iohexol if it is intended to create a high, relatively short-lived, enhancement of a tumor through intratumoral injection.

Other radiodense compositions that have similar characteristics include liposomal compositions containing equal amounts of water soluble small radiodense materials inside and outside the liposome. The size of the liposome can be varied as well as the composition of the lipid membrane. The encapsulated material can be one of several known water soluble, small molecule radiodense materials such as iohexol, iopamidol, iomeprol, iodixanol, and ioversol.

Other radiodense compositions are micellar block co-polymers such as those described in U.S. Pat. No. 5,567,410. As described above, these micellar compositions can be enriched by the addition of water soluble small molecule radiodense materials.

### Example 4

### Dwell Times of Radiodense Compositions in Vivo

Immunologically tolerant mice were implanted with several different human neoplasms. When the tumors reached a size of 1-2 cm, radiodense compositions were injected intratumorally via a percutaneous route using a 27 gauge needle. The mice and their tumors where then serially imaged with computed tomography to determine the local pharmacokinetics of the injectates by measuring the x-ray attenuation (in HU) of regions of interest.

FIG. 3 is a plot of the temporal change of x-ray attenuation in a mouse bearing a human adenocarcinomas (LS 174T). The radiodense material was a water soluble, small molecule radiodense material (Omnipaque™ with a concentration of 350 mg I/ml as iohexol). A peak contrast of nearly 3000 HU was attained in the center of the tumor, with smaller degrees of contrast enhancement surrounding the injected area. This small molecule was rapidly cleared with values of only 2000 HU at the peak location 60 minutes later.

FIG. 4 shows a similar experiment in which the human tumor was a glioma (U87-VC2) and the intratumor injectate was NI-243. In this example, the small molecule created a peak contrast of about 1300 HU, with a rapid washout over 60 minutes. The large molecule sustained a concentration of about 500 HU for more than 1 day.

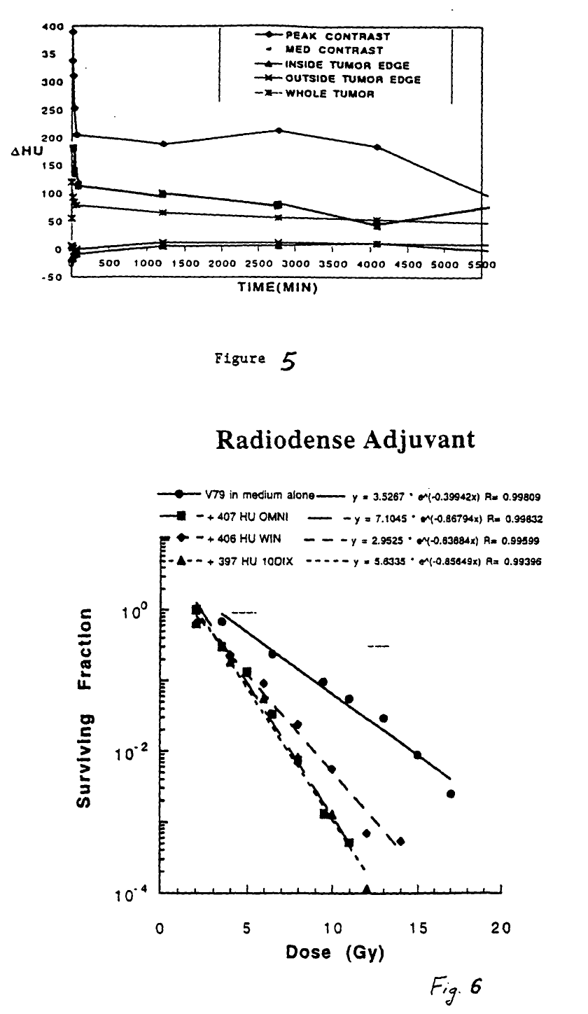

FIG. 5 shows a third experiment of this kind where the mouse was implanted with an adenocarcinoma (LS174T) and the intratumor injectate was NI-212. A smaller volume was administered and the temporal graph shows a high peak contrast that disappears with about the same clearance rate as the iohexol above, but the large molecule concentration (in the peak area) was sustained at 200 HU for about 3 days.

### Example 5

### In Vitro Evidence of Radiation Enhancement

The effect of the new radiodense compositions on cancer cells was also studied in vitro. Individual V79 Chinese Hamster Ovary cancer cells were suspended in nutrient medium in test tubes and mixed with a sufficient amount of one of several new radiodense compositions to increase the radiodensity of the cell suspension. The three radiodense compositions were Omni-350, which is a small molecule radiodense material (iohexol with an iodine concentration of 350 mg I/ml, diluted to provide 400 HU in the nutrient medium); WIN 8883, which is a large molecule radiodense material (also diluted to provide 400 HU in the nutrient medium); and iodix (iodixanol, VISIPAQUE™) which is a small molecule, water soluble nonionic dimer that is clinically available and has shown to be very safe.

As shown in FIG. 6, all three radiodense materials significantly reduced the radiation energy required to kill a particular percentage of cells. For example, at a radiation dosage of 9.0 Gray (Gy), about 1×10⁻¹ cancer cells survived in the presence of nutrient medium alone, whereas only between 1×10⁻² and 9×10⁻² cancer cells survived in the presence of the radiodense materials (with iodix and Omni being the more effective). At a radiation energy level of 12 Gy, about 8×10⁻¹ cells survived in the nutrient medium alone, while only 1×10⁻³ to 1×10⁻⁴ survived in the presence of the radiodense materials.

These results indicate that the presence of the radiodense materials significantly enhances the killing effect of radiation.

### Example 6

### In Vivo Study of Diffusion of Small and Large Radiodense Materials After Intratumoral Injection

About 200 ml of either a small molecule radiodense material (iohexol) or a large molecule radiodense material (WIN 8883) was injected directly into separate VX2 tumors growing in the thigh of a rabbit. To measure the diffusion time of the two materials in the tumor interstitium, the rabbit was euthanized and the corpus imaged using CT over the next 24 hours to measure the spatial and temporal distribution of each material.

As shown in Table 4 below, there was very little expansion of the volume of the large molecule radiodense material over 21 hours. On the other hand, the volume of the small molecule radiodense material expanded rapidly beyond the initial injection locus over the same 21 hour time period. Table 4 below shows the volume of each material over time in units of total number of intratumoral voxels containing at least 200 HU.

TABLE 4

| | Win 8883 Volume | Omni 350 Volume |
|---|---|---|
| Initial | 10420 | 12722 |
| 1.5 Hrs | 10898 | 23912 |
| 4.5 Hrs | 11826 | 22104 |
| 21.0 Hrs | 11682 | 44720 |

These results show that the large molecule radiodense material is indeed trapped within the tumor, and remains active to enhance radiation absorption, for an extended period of time of at least 21 hours.

### Example 7

### In Vivo Evidence of Radiation Enhancement

Three rabbits with VX2 tumors growing in the thigh were used to evaluate the radiation therapy effect of the new radiodense compositions and radiation administered internally. In the first rabbit, iohexol was injected systemically as a bolus in a volume of 3 ml/kg, resulting in a peak enhancement of the leaky edge of the tumor by over 50 HU. Radiation of 30 Gy was administered over 45 minutes using a delivery energy of 40 Kev from the probe tip, which was centered in the tumor. The next day, the rabbit was euthanized following the administration of Evans Blue to demarcate the leaky vasculature of the treated tumor as well as an untreated, control tumor in the opposite thigh. Histological analysis of the treated tumor revealed about 95% necrosis of viable tumor cells in the treated leg, compared with the control. All of the viable cells were located in the edge of the treated tumor and this edge of viable cells was much smaller than the rim of viable cells in the untreated tumor.

Two additional rabbits were treated with a nearly identical protocol, except that the same radiodense material was given as an intravenous bolus plus a sustaining infusion to keep the leaky portions of the tumor enhanced for the entire duration of the radiation treatment. In these two experiments, no evidence of viable tumor was seen on serial computed tomography studies over the next several days. Based on other experiments, it is known that this followup interval of several days is sufficient to identify incompletely treated VX2 tumor due to its rapid growth.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a target tissue in a patient, the method comprising
administering to the patient systemically a radiodense composition comprising a small molecule radiodense material in an amount sufficient to accumulate selectively within the target tissue compared to non-target tissue; and
inserting a radiation emitting source into the target tissue and irradiating the target tissue from within for a time and under conditions sufficient to kill cells within the target tissue.

2. A method of claim 1, wherein the target tissue is a tumor.

3. A method of claim 1, wherein the radiodense composition accumulates selectively at the outer edge of the target tissue.

4. A method of claim 1, wherein the radiation emitting source is a probe.

5. A method of claim 1, wherein the radiation emitting source comprises a radiopharmaceutical.

6. A method of claim 1, wherein the radiodense composition is administered intravenously as a bolus, followed by an infusion of the same or a different radiodense composition at a rate that equals the blood clearance rate of the radiodense composition.

7. A method of claim 1, wherein the radiodense composition comprises iohexol, iopamidol, ioversol, ioxilan, iomeprol, or iodixanol.

8. A method of claim 3, wherein the amount of the radiodense composition administered is sufficient to increase the radiation absorption of the outer edge of the target tissue by at least 10 to 200 Hounsfield units.

9. A method of claim 1, wherein the radiation has an energy of less than 140 kiloelectron volts or more than 1.02 megaelectron volts.

10. A method of claim 1, wherein the radiation has an energy of about 20 to 80 kiloelectron volts.

11. A method of claim 1, wherein the radiodense composition is linked to a targeting agent that binds specifically to the target tissue.

12. A method of treating a target tissue in a patient, the method comprising
administering to the target tissue an amount of a radiodense composition; and
irradiating the target tissue with an external radiation source emitting radiation at an energy of less than 140 kiloelectron volts or more than 1.02 megaelectron volts for a time and under conditions sufficient to kill cells within the target tissue.

13. A method of claim 12, wherein the target tissue is a tumor.

14. A method of claim 13, wherein the radiodense composition is injected directly into the tumor.

15. A method of claim 12, wherein the target tissue is diseased skin.

16. A method of claim 12, wherein the amount of the radiodense composition is sufficient to increase absorption of radiation in the target tissue by at least 10 Hounsfield units.

17. A method of claim 12, wherein the amount of the radiodense composition is sufficient to increase absorption of radiation in the target tissue by at least 200 Hounsfield units.

18. A method of claim 12, wherein the radiodense composition comprises a mixture of a small molecule radiodense material and a large molecule radiodense material.

19. A method of claim 12, wherein the radiation has an energy greater than 1.02 megaelectron volts.

20. A method of claim 12, wherein the radiation has an energy of less than 140 kiloelectron volts.

21. A method of claim 12, wherein the radiation has an energy of about 20 to 80 kiloelectron volts.

22. A method of claim 12, wherein the radiodense composition comprises iodine, barium, bismuth, boron, bromine, calcium, gold, silver, iron, manganese, nickel, gadolinium, dysprosium, tungsten, tantalum, stainless steel, or nitinol, or a combination of any one or more of the above.

23. A method of claim 12, wherein the radiodense composition comprises a radiodense material present within a small, lipid soluble molecule.

24. A method of claim 12, wherein the radiodense composition comprises a large molecule radiodense material.

25. A method of claim 12, wherein the radiodense composition has a dwell time within the target tissue of at least 3 hours.

26. A method of claim 12, wherein the radiodense composition has a dwell time within the target tissue of at least 24 hours.

27. A method of claim 12, wherein the radiodense composition is about 10 nanometers to 100 microns in size.

28. A method of claim 12, wherein the radiodense composition comprises NI-243, NI-212, or a liposome comprising iohexol.

29. A method of treating a diffuse tumor in a patient, the method comprising
administering to the patient systemically a radiodense composition comprising a small molecule radiodense material in an amount sufficient to accumulate selectively within the diffuse tumor tissue compared to non-tumor tissue; and
irradiating the body part of the patient in which the diffuse tumor is located with radiation for a time and under conditions sufficient to kill cells within the diffuse tumor.

30. A method of claim 29, wherein the diffuse tumor is a metastatic tumor.

31. A method of claim 29, wherein the radiodense composition accumulates selectively at the outer edge of the tumor and enters and accumulates within the tumor tissue.

32. A method of claim 29, wherein the radiodense composition is administered intravenously as a bolus, followed by an infusion of the same or a different radiodense composition at a rate that equals the blood clearance rate of the radiodense composition.

33. A method of claim 29, wherein the radiodense composition comprises iohexol, iopamidol, ioversol, ioxilan, iomeprol, or iodixanol.

34. A method of claim 29, wherein the radiation has an energy of less than 140 kiloelectron volts or more than 1.02 megaelectron volts.

35. A method of claim 29, wherein the radiodense composition is linked to a targeting agent that binds specifically to the target tissue.

36. A method of claim 29, wherein the radiation has an energy of greater than 1.02 megaelectron volts.

37. A method of claim 29, wherein the radiodense composition is a particle having ranging in size from 30 to 300 nanometers.

38. A method of claim 12, wherein the radiodense material is administered to the target tissue in a stent implanted within or adjacent to the target tissue.

39. A method of claim 12, wherein the target tissue is a lymph node.
